(19) United States
(12) Patent Application Publication
(10) Pub. No.: US 2009/0226408 A1
(43) Pub. Date: Sep. 10, 2009

(54) ANTI-FATIGUE COMPOSITION

(75) Inventors: Hideyuki Kishida, Hyogo (JP); Taizo Kawabe, Hyogo (JP); Kazunori Hosoe, Hyogo (JP)

Correspondence Address: SUGHRUE MION, PLLC, 2100 PENNSYLVANIA AVENUE, N.W., SUITE 800, WASHINGTON, DC, 20037, US

(73) Assignee: KANEKA CORPORATION, OSAKA (JP)

(21) Appl. No.: 11/993,743

(22) PCT Filed: Jun. 21, 2006
(86) PCT No.: PCT/JP2006/312415

§ 371 Date: Jan. 13, 2009

(30) Foreign Application Priority Data
Jun. 24, 2005 (JP) 2005-184463

(51) Int. Cl. A61K 38/44; A61K 31/122; A61K 31/385; A61K 38/45; A61P 43/00

(52) U.S. Cl. 424/94.4; 424/94.1; 514/689; 514/440; 424/94.5

(57) ABSTRACT

A method of preventing or treating fatigue in a subject mammal comprises administering an effective amount of a composition comprising (a) reduced coenzyme Q represented by the following formula (1):

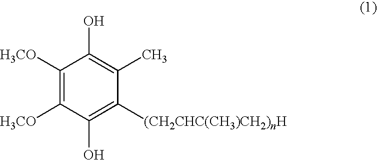

wherein n is an integer of 1 to 12, and (b) lipoic acid or a derivative thereof, to the mammal.

### TECHNICAL FIELD

The present invention relates to an anti-fatigue composition comprising a reduced coenzyme Q represented by the following formula (1):

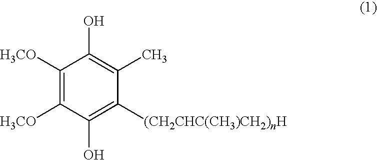

wherein n is an integer of 1 to 12, and lipoic acid or a derivative thereof as active ingredients. Here, the anti-fatigue composition refers to a composition which can achieve recovery from or prevent physical fatigue due to exercise, or which can improve physical fatigue during or after sickness, a tendency toward easily getting tired because of aging, and the like.

### BACKGROUND ART

Heretofore, a number of substances exhibiting an effect of alleviating fatigue have been reported. Lipoic acid and derivatives thereof are among such substances, and so are oxidized coenzyme Q and reduced coenzyme Q.

Coenzyme Q is an essential component widely distributed in living bodies, from bacteria to mammals. In the case of human, coenzyme Q10 in which the side chain thereof has 10 repeating units is known to be the main component. Coenzyme Q is a physiological component existing as a constituent of the electron transport system of mitochondria in cells of living bodies and, through repeated oxidation and reduction in living bodies, functions as a transmitter in the electron transport system.

Coenzyme Q is known to show energy production activity, membrane stabilizing activity and antioxidation activity in living bodies, and thus can be used for wide applications. Of the coenzymes Q, oxidized coenzyme Q (also known as ubiquinone or ubidecarenone) is known to act effectively on the heart and is used for medical purposes as a congestive heart failure drug.

Reported effects of oxidized coenzyme Q include improvement of the oxygen utilization efficiency in the cardiac muscle, activation of ATP production in the cardiac muscle and improvement of cardiac function. In addition to such medical applications, the effects as nutrients and nutritional supplements like those of vitamins have been reported.

JP-A-62-59208 (Patent Document 1) discloses a tissue metabolism-activating composition which is composed of a mixture of ubiquinone and a dry yeast powder, JP-A-52-99220 (Patent Document 2) discloses improvement of symptoms of myasthenia gravis, and JP-A-52-99222 (Patent Document 3) also reports an increase in erythrocytes and the like. Further, effects of recovery from fatigue have been reported in JP-A-7-330584 (patent Document 4), JP-A-7-330593 (Patent Document 5), JP-A-10-287560 (Patent Document 6), and WO2004/066988 (Patent Document 7).

Lipoic acid is a compound having a disulfide bond and is also called thioctic acid. At the time of discovery, lipoic acid was considered as one kind of vitamin. However, it was later found that lipoic acid was bio-synthesized in animal bodies. Lipoic acid has a role of producing energy by metabolizing the nutrients of the ingested food and a role of an antioxidant in the living body. As a pharmaceutical product, lipoic acid in the form of thioctic acid or thioctic acid amide is applied “when the demand for thioctic acid increased due to severe physical fatigue” in Japan, thus attracting attention as an anti-fatigue substance. Moreover, lipoic acid has been used overseas as a therapeutic agent for diabetes.

A combination of lipoic acid and oxidized coenzyme Q is reported to be useful for improving amnesia (Patent Document 8, JP-A-2003-513039). Moreover, it has been reported that a combination of carnitine and lipoic acid with oxidized coenzyme Q as necessary enhances energy and stamina (Patent Document 9, WO2001/21208).

### Patent Document 1: JP-A-62-59208

### Patent Document 2: JP-A-52-99220

### Patent Document 3: JP-A-52-99222

### Patent Document 4: JP-A-7-330584

### Patent Document 5: JP-A-7-330593

### Patent Document 6: JP-A-10-287560

### Patent Document 7: WO2004/066988

### Patent Document 8: JP-A-2003-513039

### Patent Document 9: WO2001/21208

### DISCLOSURE OF THE INVENTION

### Problems to be Solved by the Invention

The present invention aims at providing a safe anti-fatigue composition, which is effective even at low doses.

### Means of Solving the Problems

As described above, it is known that a mixture of oxidized coenzyme Q, carnitine and lipoic acid is used as an anti-fatigue agent. However, it is not known that a combination of reduced coenzyme Q and lipoic acid alone is effective for prevention of or recovery from fatigue. The present inventors have conducted intensive studies and found that reduced coenzyme Q and lipoic acid exhibit a synergistic effect and that a combination thereof at low doses, at which they are ineffective when used singly, also expresses an anti-fatigue effect, which resulted in the completion of the invention.

Accordingly, the present invention provides the following.

[1] An anti-fatigue composition comprising
(a) reduced coenzyme Q represented by the following formula (1):

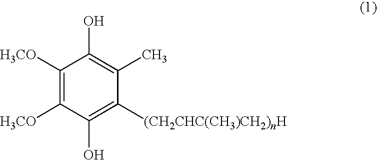

wherein n is an integer of 1 to 12, and
(b) lipoic acid or a derivative thereof.
[2] The anti-fatigue composition of [1], wherein the coenzyme Q is coenzyme Q₁₀.
[3] An anti-fatigue composition comprising
(a) reduced coenzyme Q represented by the following formula (1):

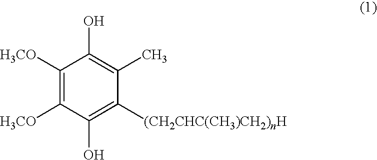

wherein n is an integer of 1 to 12, and oxidized coenzyme Q represented by the following formula (2):

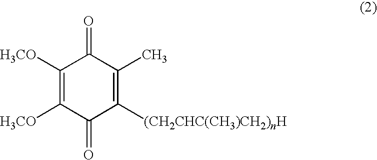

wherein n is an integer of 1 to 12, and
(b) lipoic acid or a derivative thereof.
[4] The anti-fatigue composition of [3], wherein the reduced coenzyme Q is reduced coenzyme Q₁₀ and the oxidized coenzyme Q is oxidized coenzyme Q₁₀.
[5] The anti-fatigue composition of any one of [1] to [4], wherein the lipoic acid is R-lipoic acid.
[6] A composition for a medicine or food, comprising the anti-fatigue composition of any one of [1] to [5], and a carrier acceptable as a medicine or food.
[7] The composition of [6], further comprising an antioxidant.
[8] The composition of [7], wherein the antioxidant is at least one kind selected from the group consisting of vitamin C, vitamin C derivatives, probucol, lycopene, vitamin A, vitamin A derivatives, carotenoids, vitamin B, vitamin B derivatives, flavonoids, polyphenols, glutathione, selenium, sodium thiosulfate, vitamin E, vitamin E derivatives, pycnogenol, flavangenol, pyrroloquinoline quinone, pyrroloquinoline quinone derivatives, superoxide dismutase (SOD), glutathione peroxidase, glutathione-S-transferase, glutathione reductase, catalase and ascorbate peroxidase.
[9] A method of preventing or treating fatigue in a subject mammal, which comprises administering an effective dose of the composition of any one of [1] to [8] to the mammal.
[10] A method of producing an anti-fatigue composition, which comprises mixing (a) reduced coenzyme Q represented by the aforementioned formula (1) with (b) lipoic acid or a derivative thereof.
[11] A method of producing an anti-fatigue composition, which comprises mixing (a) reduced coenzyme Q represented by the aforementioned formula (1) and oxidized coenzyme Q represented by the aforementioned formula (2) with (b) lipoic acid or a derivative thereof.
[12] An anti-fatigue composition produced by mixing (a) reduced coenzyme Q represented by the aforementioned formula (1) with (b) lipoic acid or a derivative thereof.
[13] An anti-fatigue composition produced by mixing (a) reduced coenzyme Q represented by the aforementioned formula (1) and oxidized coenzyme Q represented by the aforementioned formula (2) with (b) lipoic acid or a derivative thereof.

### EFFECT OF THE INVENTION

According to the present invention, by using reduced coenzyme Q and a lipoic acid or a derivative thereof in combination, an unexpected strong anti-fatigue effect can be afforded. In particular, it has been confirmed for the first time in the present invention that even low doses of coenzyme Q and lipoic acid or a derivative thereof, at which they are ineffective when used singly, express an anti-fatigue effect when used in combination. Conventionally, it is a general understanding that the effects of oxidized coenzyme Q and reduced coenzyme Q on the living body are identical. Therefore, it is surprising that reduced coenzyme Q exhibits such a superior effect. According to the present invention, a safe anti-fatigue pharmaceutical composition or food, which shows assured effects even in a small dose, can be provided.

### Best Mode for Embodying the Invention

Coenzyme Q exists in two forms of oxidized type and reduced type and is known to exist in living bodies, with about 40 to 90% thereof generally occurring in reduced form. The oxidized coenzyme Q can be easily obtained as a commercially available product, or by a conventionally known method such as synthesis, fermentation or extraction from a natural source and the like. The method of preparing reduced coenzyme Q is not particularly limited and for example, a method comprising obtaining coenzyme Q by a conventionally known method such as synthesis, fermentation or extraction from a natural source and concentrating the fraction of reduced coenzyme Q in eluted liquid using chromatography may be employed. In that case, it is also possible to add a common reducing agent such as sodium borohydride, sodium dithionite (sodium hydrosulfite) or ascorbic acid to the above-mentioned coenzyme Q if necessary, and after reducing the oxidized coenzyme Q contained in the above-mentioned coenzyme Q to form reduced coenzyme Q according to a conventional method, subject the same to chromatography for concentration. Reduced coenzyme Q can also be obtained by a method in which the above-mentioned reducing agent is reacted with existing high purity coenzyme Q (oxidized coenzyme Q). Alternatively, microbial cells and the like which contain reduced coenzyme Q may also be used.

In the anti-fatigue composition of the present invention, while reduced coenzyme Q serves as the active and essential ingredient coenzyme Q, a coenzyme Q, which is a mixture of reduced coenzyme Q and oxidized coenzyme Q, may be used as the active ingredient. When a coenzyme Q, which is a mixture of reduced coenzyme Q and oxidized coenzyme Q, is used, the proportion of the reduced form and the oxidized form is not particularly limited. However, since reduced coenzyme Q is an active and essential ingredient in the present invention, a higher proportion of reduced coenzyme Q relative to the entire coenzyme Q is more preferable. For example, not less than 60 wt % is preferable, not less than 80 wt % is more preferable, and not less than 95 wt % is still more preferable.

The proportion of the reduced form and the oxidized form in coenzyme Q may be generally determined by a method in which quantities of oxidized coenzyme Q and reduced coenzyme Q in a sample are measured by an HPLC system using a UV detector or the proportion can be calculated from the quantity ratio, or by a method in which the proportion of oxidized coenzyme Q and reduced coenzyme Q is calculated from a peak area according to an HPLC system to which an electrochemical detector is attached. The system with an electrochemical detector is very useful for measuring the proportion of the reduced form existing in living bodies or samples in trace amounts because the system can specifically measure oxidation-reduction substances and has a high sensitivity. Specifically, an electrochemical detector made by Shiseido Co., Ltd. was attached to an HPLC analytical system made by Shimadzu Corporation, and the measurement was conducted under the following HPLC conditions: column: YMC-Pack (ODS-A303), detection wavelength: 275 nm, mobile phase: methanol (88%), hexane (12%), flow rate: 1 ml/min.

The reduced coenzyme Q and the oxidized coenzyme Q that can be used in the present invention are those in which the number of repeating units (n in the formulas) in the side chain is 1 to 12, as represented by the above-mentioned formulas (1) and (2). Of these, those containing 10 repeating units in the side chain, i.e., reduced coenzyme Q₁₀ or oxidized coenzyme Q₁₀, can be suitably used from the viewpoint that sufficient effects can be obtained in human and many pets such as dogs.

The chemical name of lipoic acid is 1,2-dithioran-3-pentanoic acid (thioctic acid). Lipoic acid and lipoic acid derivatives can be produced by a conventionally known method such as synthesis, fermentation, extraction from a natural source, and the like. Moreover, a commercially available product can be used as is. Alternatively, yeast, other fungus, dried product thereof and food that highly contain lipoic acid or a lipoic acid derivative can also be used.

As used herein, a derivative refers to a compound obtained by a small structural modification in a certain compound. A compound in which a hydrogen atom or a specific atomic group is substituted by another atom or another atomic group is understood to be a derivative of the original compound.

Lipoic acid includes, for example, α-lipoic acid (mixture of R-form and S-form), R-lipoic acid, S-lipoic acid, and the like. Examples of the lipoic acid derivative are not particularly limited, and a preferable example is a metabolite of lipoic acid, i.e., dihydrolipoic acid. Examples of further metabolite also include lipoamide, lipoyllysin, di-6,8-bisnorlipoic acid, tetranorlipoic acid, and the like. Such lipoic acid derivatives include respective optical isomers of the derivative. Furthermore, lipoic acid derivative includes pharmaceutically acceptable salts of lipoic acid and the above-mentioned derivatives and the like. Those may be used alone or in combination of two or more kinds as the lipoic acid or the lipoic acid derivative in the present invention. Preferred are α-lipoic acid and R-lipoic acid, with more preference given to naturally occurring R-lipoic acid.

Examples of the pharmaceutically acceptable salt of lipoic acid or a derivative thereof include, when the lipoic acid derivative contains a basic moiety, salts with hydrochloric acid, sulfuric acid, methanesulfonic acid, fumaric acid, maleic acid, succinic acid, acetic acid, benzoic acid, oxalic acid, citric acid, tartaric acid, carbonic acid, or phosphoric acid. When lipoic acid and a lipoic acid derivative contain an acidic moiety, examples thereof include alkali metal salts such as sodium salt and potassium salt; alkaline earth metal salts such as calcium salt and magnesium salt; and salts formed with a suitable organic ligand, such as quaternary ammonium salt. Hereinafter, “lipoic acid and lipoic acid derivative” are collectively referred to as a “lipoic acid derivative”.

In the anti-fatigue composition of the present invention, the contents of reduced coenzyme Q and lipoic acid derivatives are not particularly limited and they may be accordingly determined depending on the commercial concept of the product.

The weight ratio of reduced coenzyme Q and lipoic acid derivative in the anti-fatigue composition of the present invention is not limited, but from the viewpoint of achieving a sufficient synergistic effect, the weight ratio is within the range 100:1 to 1:100, preferably 10:1 to 1:10, more preferably about 5:1 to about 1:5, most preferably about 1:1.

As a method for analyzing the lipoic acid derivative, a known analytical method can be used. In the present invention, an electrochemical detector (ECD) was connected to an HPLC system, and the lipoic acid derivative was separated using a Hypersil C18 column (125 mm×4.6 mmi.d, particle size 3 μm) and the mobile phase of a phosphate buffer (50 mM KH₂PO₄/H₃PO₄, pH 2.7) and acetonitrile (65:35, v/v). Prior to the HPLC analysis, the analysis sample was suction filtered through a filter with pore diameter of 0.45 μm. In the HPLC analysis, the flow rate of the mobile phase was set to 0.7 ml/min.

The dosage form of the anti-fatigue composition of the present invention is not particularly limited, and it may be an oral preparation or a preparation applied directly to the skin. The oral preparation may be a powder preparation or may be formed into a granular preparation by adding a binder. In addition, such powder or granules may be packed into capsules to prepare a capsule preparation.

Further, natural oil, oily higher fatty acid, higher fatty acid monoglyceride, medium-chain triglyceride (MCT), a surfactant or a mixture thereof may be added, and these oily substances may be packed as is to prepare soft capsules. In that case, capsules composed mainly of gelatin or capsules composed mainly of other water soluble polymer substances may be used. In addition, such capsules include microcapsules. Alternatively, the composition may be formed into liquid to prepare a drinkable preparation.

Pharmaceutically acceptable carriers (preparation ingredients) other than the above-mentioned reduced coenzyme Q, lipoic acid derivatives, and oxidized coenzyme Q to be added or contained as necessary, may be suitably added to and mixed with the anti-fatigue composition of the present invention by a conventional method. Examples thereof include, but are not limited to, an excipient, a disintegrant, a lubricant, a binder, an antioxidant, a colorant, an anti-agglomeration agent, an absorption promoter, a solubilizing agent, a stabilizer and the like.

Examples of such excipient include, but not limited to, saccharose, lactose, glucose, cornstarch, mannitol, crystalline cellulose, calcium phosphate, calcium sulfate and the like. Examples of such disintegrant include, but not limited to, starch, agar, calcium citrate, calcium carbonate, sodium hydrogen carbonate, dextrin, crystalline cellulose, carboxymethyl cellulose, tragacanth and the like.

Examples of such lubricant include, but not limited to, talc, magnesium stearate, polyethylene glycol, silica, hydrogenated vegetable oil and the like. Examples of such binder include, but not limited to, ethyl cellulose, methyl cellulose, hydroxypropylmethyl cellulose, tragacanth, shellac, gelatin, gum arabic, polyvinylpyrrolidone, polyvinyl alcohol, polyacrylic acid, polymethacrylic acid, sorbitol and the like.

Examples of such antioxidant include, but not limited to, ascorbic acid, tocopherol, vitamin A, 1-carotene, sodium bisulfite, sodium thiosulfate, sodium pyrosulfite, citric acid and the like.

The above-mentioned colorant is not particularly limited and those permitted to be added to medicines and food products may be used.

Examples of such anti-agglomeration agent include, but not limited to, stearic acid, talc, light anhydrous silicic acid, hydrous silicon dioxide and the like.

Examples of such absorption promoter include, but not limited to, surfactants such as higher alcohols, higher fatty acids, glycerine fatty acid esters, sucrose fatty acid esters, sorbitan fatty acid esters, polyoxyethylenes, sorbitan fatty acid esters and polyglycerine fatty acid esters.

Examples of such solubilizing agent include, but not limited to, organic acids such as fumaric acid, succinic acid and malic acid. Examples of such stabilizer include, but not limited to, benzoic acid, sodium benzoate ethyl parahydroxybenzoate and the like.

When applying directly to the skin, the dosage form is not particularly limited, and examples of dosage form include those prepared by dissolving, or mixing and dispersing the composition of the present invention in a suitable base, into cream, paste, jelly, gel, emulsion or liquid (ointment, liniment, lotion, cream, spray, etc.); those obtained by dissolving, or mixing and dispersing the composition of the present invention in a suitable base and spreading the resultant on a support (poultices, etc.); and those obtained by dissolving, or mixing and dispersing the composition of the present invention in an adhesive and spreading the resultant on a support (plaster, tape, etc.). As the base and adhesive, those usually used for medicines and cosmetics may be used according to need within the range in which the effect of the present invention is not damaged.

The anti-fatigue composition of the present invention may contain an antioxidant such as an antioxidant material, antioxidant enzyme or the like. Suitable examples of such antioxidants include, but not limited to, vitamin E, vitamin E derivatives, vitamin C, vitamin C derivatives, probucol, lycopene, vitamin A, vitamin A derivatives, carotenoids, vitamin B, vitamin B derivatives, flavonoids, polyphenols, glutathione, pyrroloquinoline quinone, pyrroloquinoline quinine derivatives, pycnogenol, flavangenol, sodium thiosulfate and selenium. In addition, antioxidant enzymes may be used as an anti-oxidant. Suitable examples of antioxidant enzymes include, but not limited to, superoxide dismutase (SOD), glutathione peroxidase, glutathione-S-transferase, glutathione reductase, catalase and ascorbate peroxidase. The above-mentioned antioxidants (including antioxidant enzymes) may be used alone, or in a mixture of two or more kinds thereof.

The anti-fatigue composition of the present invention may also contain other revitalizing ingredients. Suitable examples of revitalizing ingredients include, but not limited to, creatine, taurine, vitamin B₁, vitamin B derivatives, amino acids and the like. These may be used alone, or two or more kinds of these may be mixed. By mixing these ingredients with the coenzyme Q and the lipoic acid derivative in the present invention, further additive or synergistic effects can be expected.

The anti-fatigue composition of the present invention may also contain a supplemental nutrient. Examples of supplemental nutrient include, but not limited to, amino acids, metal ions, saccharides, proteins, fatty acids, vitamins and the like.

When the anti-fatigue composition of the present invention is processed into food, examples of the form thereof include, but are not limited to, edible fat and oil composition, cooking oil, spray oil, butter, margarine, shortening, whipping cream, concentrated milk, whiteners, dressings, pickle liquids, breads, cakes, pies, cookies, Japanese confectioneries, snacks, fried snacks, chocolates and chocolate confectioneries, rice confectioneries, roux, sauce, basting, toppings, ice creams, noodles, bread mix, fried food, processed meat products, fish paste products, frozen food such as frozen entrees, frozen meat and frozen vegetables, rice, jam, cheese, cheese food, cheese-like food, chewing gums, candies, fermented milk, canned food, drinks and the like.

By applying the anti-fatigue composition of the present invention directly to the skin, muscle fatigue can be alleviated. In that case, the composition may also contain a substance having antiinflammatory effects. Such substances having antiinflammatory effects are not particularly limited and include steroid, salicylic acid and derivatives thereof, arylacetic acid and derivatives thereof, propionic acid and derivatives thereof, fenamic acid and derivatives thereof, pyrazolone and derivatives thereof, oxicam and derivatives thereof, non-acidic antiinflammatory agents and the like. Such substances may be used alone or in combination of two or more kinds thereof.

Examples of steroids include prednisolone valerate acetate, amcinonide, diflucortolone valerate, dexamethasone valerate, clobetasol propionate, diflorasone diacetate, dexamethasone propionate, betamethasone dipropionate, difluprednate, fluocinonide, halcinonide, budesonide, hydrocortisone butyrate propionate, betamethasone valerate, beclometasone dipropionate, fluocinolone acetonide, triamcinolone acetonide, flumetasone pivalate, hydrocortisone butyrate, clobetasone butyrate, alclometasone dipropionate, dexamethasone, methylprednisolone acetate, prednisolone and hydrocortisone acetate, and the like.

Examples of salicylic acid derivatives include aspirin and derivatives thereof, diflunisal and the like. Examples of arylacetic acid derivatives include indomethacin, diclofenac, sulindac, nabumetone, proglumetacin, indomethacinfarnesyl, etodolac and the like. Examples of propionic acid derivatives include ibuprofen, naproxen, flurbiprofen, fenoprofen, tiaprofen, pranoprofen, loxoprofen, alminoprofen and the like. Examples of fenamic acid derivatives include mefenamic acid, tolfenamic acid and the like.

Examples of pyrazolone derivatives include phenylbutazone, oxyphenbutazone and the like. Examples of oxicam derivatives include piroxicam, tenoxicam, ampiroxicam and the like. Examples of non-acidic antiinflammatory agent include epirizole, tiaramide, emorfazone and the like. By further mixing these ingredients with the anti-fatigue composition containing reduced coenzyme Q of the present invention and a lipoic acid derivative, additive or synergistic effects can be expected.

The “anti-fatigue effect” in the present invention means an effect of preventing, treating, recovering from, improving or curing fatigue. The administration subjects of the anti-fatigue composition of the present invention includes, human, and other mammals such as dog, cat and the like. By administering an effective dose of the anti-fatigue composition of the present invention to the subject mammal, the fatigue of the subject mammal can be prevented or treated. The “treatment” as used herein means recovery, improvement or curing.

The “effective dose” used herein means an amount containing the active ingredients capable of providing an anti-fatigue effect to the administration subject. In this case, the anti-fatigue effect may be objective (that is, measurable by some test or marker) or subjective (that is, felt effective by the subject). The dose level of the aforementioned active ingredients and frequency of administration of the specific combination vary depending on the potency of each active ingredient to be used, the metabolic stability and duration of the action of each active ingredient, the species, age, body weight, general health condition, and sex of a subject person (subject animal), diet, mode and time of administration, clearance rate, combination of drugs, severity of the condition to be treated, and the type of treatment the subject person (subject animal) experienced, and the like.

The determination of the effective dose is within the ordinary technique of a person skilled in the art, and a person skilled in the art can determine the dose based on his/her experimentation or experience. Examples of the effective dose in the present invention include a combination of about 0.01 mg-1000 mg/kg body weight/day of reduced coenzyme Q and about 0.01-1000 mg/kg body weight/day of lipoic acid derivative; preferably about 0.01-500 mg/kg body weight/day of reduced coenzyme Q and about 0.01-500 mg/kg body weight/day of lipoic acid derivative, and particularly about 0.1-500 mg/kg body weight/day of reduced coenzyme Q and about 0.1-500 mg/kg body weight/day of lipoic acid derivative. The above-mentioned effective dose can be administered singly or in multiple portions, generally by an oral route. For example, non-oral administration route such as dermal administration, enteric administration and the like can be employed.

The composition of the present invention can also be considered as a synergistic composition. By the term “synergistic composition” used here is meant a composition which exhibits, due to the interaction between the reduced coenzyme Q and the lipoic acid derivative, an activity greater than that expected from the action observed when each is separately applied, i.e., an additive activity. Example of the synergistically effective dose of the synergistic composition is a dose smaller than the aforementioned effective doses. For example, about 0.01 mg-300 mg/kg body weight/day of reduced coenzyme Q and about 0.01-300 mg/kg body weight/day of lipoic acid derivative, preferably about 0.01-200 mg/kg body weight/day of reduced coenzyme Q and about 0.01-200 mg/kg body weight/day of lipoic acid derivative, and particularly preferably about 0.1-200 mg/kg body weight/day of reduced coenzyme Q and about 0.1-200 mg/kg body weight/day of lipoic acid derivative.

The determination of the effective dose of each active ingredient contained in the composition of the present invention is within the ordinary technique of a person skilled in the art, and a person skilled in the art can determine the dose based on his/her experimentation or experience. Examples of the effective dose is 0.01-1000 mg of reduced coenzyme Q in the composition and about 0.01-1000 mg of lipoic acid derivative in the composition; preferably about 0.01-500 mg of reduced coenzyme Q in the composition and about 0.01-500 mg of lipoic acid derivative in the composition, and particularly preferably about 0.1-500 mg of reduced coenzyme Q in the composition and about 0.1-500 mg of lipoic acid derivative in the composition.

The present invention further provides a combined preparation in which the first active ingredient (reduced coenzyme Q) and the second active ingredient (lipoic acid derivative) in a synergistically effective dose are respectively formulated into separate units, such as capsule, tablet and pill, and combined with each other. Moreover, the present invention also provides a commercial product containing the combined preparation and a written instruction to the effect that the preparation should be applied simultaneously or sequentially as necessary. Of course, the present invention does not exclude an embodiment in which the reduced coenzyme Q and the lipoic acid derivative are contained and used in a single preparation, or such commercial products.

The anti-fatigue composition of the present invention can be produced by a method comprising obtaining reduced coenzyme Q by an existing method and mixing the reduced coenzyme Q and a lipoic acid derivative. Alternatively, the oxidized coenzyme Q and lipoic acid derivative are mixed and, at some time point in the production process, for example, before or after mixing the oxidized coenzyme Q and lipoic acid, at least a part of the oxidized coenzyme Q may be converted to reduced coenzyme Q.

The form of the product and the method and mode of preservation of the product of the anti-fatigue composition containing reduced coenzyme Q and a lipoic acid derivative of the present invention can be appropriately determined according to the product design and the purpose of use of the anti-fatigue composition.

### EXAMPLES

Hereinafter, the present invention is described in more detail referring to Examples, Formulation Examples and Preparation Examples, but the present invention is not limited to these Examples.

### Production Example 1

### Preparation of Reduced Coenzyme Q₁₀

To 1000 g of ethanol were added 100 g of oxidized coenzyme Q₁₀ (purity 99.4%) and 60 g of L-ascorbic acid. The mixture was stirred at 78° C. to conduct a reduction reaction. After 30 hours, the mixture was cooled to 50° C. and with maintaining the temperature, 330 g of ethanol and 70 g of water were added thereto. With stirring the ethanol solution (containing 100 g of reduced coenzyme Q₁₀), the solution was cooled to 2° C. at a cooling rate of 10° C./hour to obtain white slurry. The obtained slurry was filtrated under reduced pressure and the resulting wet crystal was washed with cold ethanol, cold water and cold ethanol in that order (temperature of cold solvent used for washing: 2° C.), and further, the wet crystal was dried under reduced pressure (20 to 40° C., 1 to 30 mmHg) to obtain 97 g of reduced coenzyme Q₁₀ (containing 2% oxidized coenzyme Q₁₀) white dry crystals. All procedures except drying under reduced pressure were carried out in a nitrogen atmosphere.

### Production Example 2

100 g of oxidized coenzyme Q₁₀ was dissolved in 1000 g of a heptane solution at 25° C. With stirring, an aqueous solution obtained by adding 1000 g of water to 100 g of sodium hydrosulfite (purity 75% or higher) as a reducing agent and dissolving the same therein was gradually added to the above-mentioned heptane solution to conduct a reduction reaction while controlling to 25° C., pH 4 to 6. After 2 hours, the aqueous phase was removed from the reaction mixture and the heptane phase was washed with 1000 g of deaerated saturated saline 6 times. The heptane phase was subjected to solvent replacement under reduced pressure and a 7% (w/w) ethanol solution of reduced coenzyme Q₁₀ (containing 100 g of reduced coenzyme Q₁₀) of a temperature of 50° C. was prepared. To the ethanol solution was added 50 g of water and with stirring, the solution was cooled to 2° C. at a cooling rate of 10° C./hour to allow crystal precipitation. The obtained slurry was filtrated under reduced pressure and the resulting wet crystal was washed with cold ethanol, cold water and cold ethanol in that order (temperature of cold solvent used for washing: 2° C.), and further, the wet crystal was dried under reduced pressure (20 to 40° C., 1 to 30 mmHg) to give 97 g of reduced coenzyme Q₁₀ (containing 2% oxidized coenzyme Q₁₀) white dry crystals. All the operations except vacuum drying were performed in a nitrogen atmosphere.

### Example 1

### Treadmill Test Using Young Rat

Using Sprague-Dawley male rats (7 to 10 week old, n=20), anti-fatigue effects brought about by reduced coenzyme Q₁₀ alone (about 2% of oxidized coenzyme Q₁₀ included), lipoic acid alone, and combination use of reduced coenzyme Q₁₀ and lipoic acid were evaluated with a treadmill. In the test, rats were run at a speed of 10 m/min using a treadmill apparatus (model NK-73-4 made by Natume Seisakusho Co., Ltd.), and the speed was increased stepwise, by 5 m/min every 3 min, and the time when the rats became unable to run (maximum running time) was measured.

As substances to be tested, 0.5% sodium carboxymethylcellulose (CMC-Na) aqueous solutions of reduced coenzyme Q₁₀ (about 2% oxidized coenzyme Q₁₀ included) or lipoic acid (α-lipoic acid (manufactured by Tokyo Chemical Industry Co., Ltd.)) were each prepared and the solutions were orally administered to rats at a dose of 100 mg/kg for reduced coenzyme Q₁₀ or at a dose of 30 mg/kg for lipoic acid. Rats were divided into four groups of a solvent administered group, reduced coenzyme Q₁₀ only administered group, lipoic acid only administered group, and a reduced coenzyme Q₁₀ and lipoic acid combined group (reduced coenzyme Q₁₀ and lipoic acid were administered almost simultaneously). The maximum running time was measured before and three hours after the administration and an extension of the maximum running time (after the sample administration relative to before the administration) was calculated. A 0.5% CMC-Na aqueous solution was administered to the control group. The maximum running time values are shown in Table 1.

TABLE 1

| | | | | Reduced |
|---|---|---|---|---|
| | | Reduced | | coenzyme Q₁₀ + |
| | Control | coenzyme Q₁₀ | Lipoic acid | lipoic |
| | group | group | group | acid |
| Average | −11.7 | 17.5 | 7.3 | 60.9 |
| (second) | | | | |
| S.D. | 25.0 | 41.0 | 28.0 | 60.7 |
| Test | | | | P < 0.001 |

In the control group, the difference in the maximum running times before and after the administration of the test substance is −11.7±25 seconds, which suggested that the maximum running time slightly decreased. In the lipoic acid or reduced coenzyme Q₁₀ only administered group, no significant extension of the maximum running time of the rats were observed before and after the administration, but in the reduced coenzyme Q₁₀ and lipoic acid administered group, the maximum running times of the rats significantly increased compared to those of the control group. In other words, by combining reduced coenzyme Q₁₀ and lipoic acid, an anti-fatigue effect which was not apparent when they are individually used is now clearly shown, proving that the combination of these substances generates a synergistic anti-fatigue effect.

### Formulation Example 1

Olive oil was heated to 60° C. and coenzyme Q₁₀ (reduced coenzyme Q₁₀:oxidized coenzyme Q₁₀=85:15) and α-lipoic acid melted also at 60° C. were added thereto. Vitamin E was gradually added to the mixture until homogeneous and soft capsules were prepared according to a conventional method. Soft capsules containing 20 mg of coenzyme Q₁₀ (17 mg of reduced coenzyme Q₁₀) and 20 mg of α-lipoic acid per capsule were prepared.

| | | |
|---|---|---|
| Coenzyme Q₁₀ | 20 | parts by weight |
| (reduced coenzyme Q₁₀:oxidized coenzyme | | |
| Q₁₀ = 85:15) | | |
| α-Lipoic acid | 20 | parts by weight |
| Vitamin E | 15 | parts by weight |
| Olive oil | 350 | parts by weight |

### Formulation Example 2

Olive oil was heated to 60° C. and coenzyme Q₁₀ (reduced coenzyme Q₁₀:oxidized coenzyme Q₁₀=85:15) and R-lipoic acid melted also at 60° C. were added thereto. Vitamin E was gradually added to the mixture until homogeneous and soft capsules were prepared according to a conventional method. Soft capsules containing 20 mg of coenzyme Q₁₀ (17 mg of reduced coenzyme Q₁₀) and 20 mg of R-lipoic acid per capsule were prepared.

| | | |
|---|---|---|
| Coenzyme Q10 | 20 | parts by weight |
| (reduced coenzyme Q10:oxidized coenzyme | | |
| Q10 = 85:15) | | |
| R-Lipoic acid | 20 | parts by weight |
| Vitamin E | 15 | parts by weight |
| Olive oil | 350 | parts by weight |

### Formulation Example 3

Coenzyme Q₁₀ (reduced coenzyme Q₁₀:oxidized coenzyme Q₁₀=85:15) and α-lipoic acid were added to a suitable amount of acetone and the mixture was then adsorbed on crystalline cellulose (fine powder) followed by drying. The resultant was mixed with cornstarch and formed into powder according to a conventional method.

| | |
|---|---|
| Coenzyme Q₁₀ | 10 parts by weight |
| (reduced coenzyme Q₁₀:oxidized coenzyme | |
| Q₁₀ = 85:15) | |
| α-Lipoic acid | 10 parts by weight |
| Crystalline cellulose | 40 parts by weight |
| Cornstarch | 55 parts by weight |

### Formulation Example 4

Coenzyme Q₁₀ (reduced coenzyme Q₁₀:oxidized coenzyme Q₁₀=85:15) and R-lipoic acid were added to a suitable amount of acetone and the mixture was then adsorbed on crystalline cellulose (fine powder) followed by drying. The resultant was mixed with cornstarch and formed into powder according to a conventional method.

| | |
|---|---|
| Coenzyme Q₁₀ | 10 parts by weight |
| (reduced coenzyme Q₁₀:oxidized coenzyme | |
| Q₁₀ = 85:15) | |
| R-Lipoic acid | 10 parts by weight |
| Crystalline cellulose | 40 parts by weight |
| Cornstarch | 55 parts by weight |

### Formulation Example 5

Coenzyme Q₁₀ (reduced coenzyme Q₁₀:oxidized coenzyme Q₁₀=85:15) and α-lipoic acid were added to a suitable amount of acetone and the mixture was then adsorbed on crystalline cellulose (fine powder) followed by drying. Cornstarch, lactose, carboxymethylcellulose and magnesium stearate were mixed thereto and an aqueous polyvinylpyrrolidone solution was then added thereto as a binder to form granules according to a conventional method. After adding talc thereto as a lubricant and mixing, tablets containing 20 mg of coenzyme Q₁₀ (17 mg of reduced coenzyme Q₁₀) and 20 mg of α-lipoic acid per tablet were prepared.

| | |
|---|---|
| Coenzyme Q₁₀ | 20 parts by weight |
| (reduced coenzyme Q₁₀:oxidized coenzyme | |
| Q₁₀ = 85:15) | |
| α-Lipoic acid | 20 parts by weight |
| Cornstarch | 25 parts by weight |
| Lactose | 15 parts by weight |
| Carboxymethylcellulose | 10 parts by weight |
| Crystalline cellulose | 40 parts by weight |
| Polyvinylpyrrolidone | 5 parts by weight |
| Magnesium stearate | 3 parts by weight |
| Talc | 10 parts by weight |

### Formulation Example 6

Coenzyme Q₁₀ (reduced coenzyme Q₁₀:oxidized coenzyme Q₁₀=85:15) and R-lipoic acid were added to a suitable amount of acetone and the mixture was then adsorbed on crystalline cellulose (fine powder) followed by drying. Cornstarch, lactose, carboxymethylcellulose and magnesium stearate were mixed thereto and an aqueous polyvinylpyrrolidone solution was then added thereto as a binder to form granules according to a conventional method. After adding talc thereto as a lubricant and mixing, tablets containing 20 mg of coenzyme Q₁₀ (17 mg of reduced coenzyme Q₁₀) and 20 mg of R-lipoic acid per tablet were prepared.

| | |
|---|---|
| Coenzyme Q₁₀ | 20 parts by weight |
| (reduced coenzyme Q₁₀:oxidized coenzyme | |
| Q₁₀ = 85:15) | |
| R-Lipoic acid | 20 parts by weight |
| Cornstarch | 25 parts by weight |
| Lactose | 15 parts by weight |
| Carboxymethylcellulose | 10 parts by weight |
| Crystalline cellulose | 40 parts by weight |
| Polyvinylpyrrolidone | 5 parts by weight |
| Magnesium stearate | 3 parts by weight |
| Talc | 10 parts by weight |

### Formulation Example 7

After granulating the following ingredients according to a conventional method, the resultant was packed in hard gelatin capsules. Capsules containing 20 mg of coenzyme Q₁₀ (17 mg of reduced coenzyme Q₁₀) and 20 mg of α-lipoic acid per capsule were obtained.

| | |
|---|---|
| Coenzyme Q₁₀ | 20 parts by weight |
| (reduced coenzyme Q₁₀:oxidized coenzyme | |
| Q₁₀ = 85:15) | |
| α-Lipoic acid | 20 parts by weight |
| Crystalline cellulose | 40 parts by weight |
| Cornstarch | 20 parts by weight |
| Lactose | 62 parts by weight |
| Magnesium stearate | 2 parts by weight |
| Polyvinylpyrrolidone | 3 parts by weight |

### Formulation Example 8

After granulating the following ingredients according to a conventional method, the resultant was packed in hard gelatin capsules. Capsules containing 20 mg of coenzyme Q₁₀ (17 mg of reduced coenzyme Q₁₀) and 20 mg of R-lipoic acid per capsule were obtained.

| | |
|---|---|
| Coenzyme Q₁₀ | 20 parts by weight |
| (reduced coenzyme Q₁₀:oxidized coenzyme | |
| Q₁₀ = 85:15) | |
| R-Lipoic acid | 20 parts by weight |
| Crystalline cellulose | 40 parts by weight |
| Cornstarch | 20 parts by weight |
| Lactose | 62 parts by weight |
| Magnesium stearate | 2 parts by weight |
| Polyvinylpyrrolidone | 3 parts by weight |

### Formulation Example 9

A cream of the following formulation containing coenzyme Q₁₀ (reduced coenzyme Q₁₀:oxidized coenzyme Q₁₀=85:15) and R-lipoic acid was prepared according to a conventional method.

| | | |
|---|---|---|
| Coenzyme Q₁₀ | 10 | parts by weight |
| (reduced coenzyme Q₁₀:oxidized coenzyme | | |
| Q₁₀ = 85:15) | | |
| R-lipoic acid | 10 | parts by weight |
| Glycerolsorbitan fatty acid ester | 60 | parts by weight |
| Microcrystalline wax | 10 | parts by weight |
| Olive oil | 30 | parts by weight |
| Liquid paraffin | 180 | parts by weight |
| Magnesium stearate | 10 | parts by weight |
| Propylene glycol | 37 | parts by weight |
| Magnesium sulfate | 7 | parts by weight |
| Deionized water | 645 | parts by weight |

### Formulation Example 10

A cream of the following formulation containing coenzyme Q₁₀ (reduced coenzyme Q₁₀:oxidized coenzyme Q₁₀=85:15) and R-lipoic acid was prepared according to a conventional method.

| | | |
|---|---|---|
| Coenzyme Q₁₀ | 0.1 | part by weight |
| (reduced coenzyme Q₁₀:oxidized coenzyme | | |
| Q₁₀ = 85:15) | | |
| R-Lipoic acid | 0.1 | part by weight |
| Glycerolsorbitan fatty acid ester | 60 | parts by weight |
| Microcrystalline wax | 10 | parts by weight |
| Olive oil | 40 | parts by weight |
| Liquid paraffin | 180 | parts by weight |
| Magnesium stearate | 10 | parts by weight |
| Propylene glycol | 37 | parts by weight |
| Magnesium sulfate | 7 | parts by weight |
| Deionized water | 655 | parts by weight |

### Formulation Example 11

A hydrophilic skin ointment of the following formulation containing coenzyme Q₁₀ (reduced coenzyme Q₁₀:oxidized coenzyme Q₁₀=85:15) and R-lipoic acid according to a known method.

| | | |
|---|---|---|
| Hydrophilic ointment | 94 | parts by weight |
| Coenzyme Q₁₀ | 1 | part by weight |
| (reduced coenzyme Q₁₀:oxidized coenzyme | | |
| Q₁₀ = 85:15) | | |
| R-Lipoic acid | 1 | part by weight |
| Indomethacin | 1 | part by weight |
| Ascorbyl stearate | 3 | part by weight |

### Formulation Example 12

A W/O cream of the following formulation containing coenzyme Q₁₀ (reduced coenzyme Q₁₀:oxidized coenzyme Q₁₀=85:15) and R-lipoic acid was prepared according to a known method.

| Glycerolsorbitan fatty acid ester | 6.000 | parts by weight |
|---|---|---|
| Microcrystalline wax | 1.000 | part by weight |
| Olive oil | 3.000 | parts by weight |
| Liquid paraffin | 18.000 | parts by weight |
| Magnesium stearate | 1.000 | part by weight |
| Propylene glycol | 3.700 | parts by weight |
| Magnesium sulfate MgSO₄•7H₂O) | 0.700 | part by weight |
| Coenzyme Q₁₀ | 1.000 | part by weight |
| (reduced coenzyme Q₁₀:oxidized coenzyme | | |
| Q₁₀ = 85:15) | | |
| R-Lipoic acid | 1.000 | part by weight |
| Indomethacin | 1.000 | part by weight |
| Deionized water | 100.000 | parts by weight |

### Formulation Example 13

A W/O emulsion (dermal formulation) of the following formulation containing coenzyme Q₁₀ (reduced coenzyme Q₁₀:oxidized coenzyme Q₁₀=85:15) and R-lipoic acid was prepared according to a known method.

| Polyoxyethyleneglycerolsorbitan | 3.600 | parts by weight |
|---|---|---|
| fatty acid ester | | |
| Polyoxyethylenefatty acid ester | 1.400 | parts by weight |
| Cetearyl alcohol | 2.000 | parts by weight |
| Mineral oil, GP9 | 20.000 | parts by weight |
| Paraben mixture | appropriately | |
| Magnesium sulfate (MgSO₄•7H₂O) | 0.700 | part by weight |
| Coenzyme Q₁₀ | 1.000 | part by weight |
| (reduced coenzyme Q₁₀:oxidized coenzyme | | |
| Q₁₀ = 85:15) | | |
| R-Lipoic acid | 1.000 | part by weight |
| Calcium chloride (CaCl₂) | 0.85 | part by weight |
| Vitamin E | 1.000 | part by weight |
| Indomethacin | 1.000 | part by weight |
| Deionized water | 100.000 | parts by weight |

### Formulation Example 14

A W/O lotion of the following formulation containing coenzyme Q₁₀ (reduced coenzyme Q₁₀:oxidized coenzyme Q₁₀=85:15) and R-lipoic acid was prepared according to a known method.

| | | |
|---|---|---|
| Glycerolsorbitan fatty acid ester | 1.300 | parts by weight |
| Polyoxyethylene fatty acid ester | 3.700 | parts by weight |
| Neutral oil | 6.000 | parts by weight |
| Liquid paraffin, GP9 | 14.000 | parts by weight |
| Propylene glycol | 3.800 | parts by weight |
| Magnesium sulfate (MgSO₄•7H₂O) | 0.700 | part by weight |
| Lipoic acid | 1.500 | parts by weight |
| Coenzyme Q₁₀ | 1.000 | part by weight |
| R-Lipoic acid | 1.000 | part by weight |
| Ascorbic acid | 2.000 | parts by weight |
| Deionized water | 100.000 | parts by weight |

### Preparation Example 1

### Health Drink

Water was added to coenzyme Q₁₀ (5 g, reduced coenzyme Q₁₀:oxidized coenzyme Q₁₀=85:15), α-lipoic acid (5 g), saccharide (150 g), honey (15 g), ascorbic acid (1 g), citric acid (0.3 g), aspartame (2 g), and a suitable amount of flavoring agent to a total amount of 1000 ml. The mixture was sterilized at 95° C. for 20 min. Each 100 ml thereof was aseptically filled in a bottle to give a health drink.

### Preparation Example 2

### Margarine

In a 5 L beaker, a hydrogenated cottonseed oil composition (96 parts), coenzyme Q₁₀ (2 parts, reduced coenzyme Q₁₀:oxidized coenzyme Q₁₀=85:15) and α-lipoic acid (2 parts) were dissolved by gently stirring with heating at 60-65° C. to give a fat and oil composition. The obtained fat and oil composition (83.5 parts) and water (16.5 parts) were emulsified by stirring in an emulsion tank at 60-65° C. for 15 min and rapidly cooled to and kneaded at 15° C. The margarine prepared in this manner did not have any problem in flavor and physical properties.

### Preparation Example 3

### Bread

To flour (70 parts) were added yeast (2 parts), yeast food (0.1 part) and then water (40 parts). The mixture was kneaded in a mixer to give an intermediate dough. After 4 hr of preliminary fermentation, flour (30 parts), sugar (5 parts), margarine (6 parts) containing coenzyme Q₁₀ and α-lipoic acid, prepared in Preparation Example 2, table salt (2 parts), skim milk (3 parts) and water (23 parts) were further added to give a final dough. Bread was made in a final fermentation at 38° C. for 50 min and baking at 180° C., 35 min. The obtained bread had excellent flavor and color.

1.-13. (canceled)

14. A method of preventing or treating fatigue in a subject mammal, which comprises administering an effective amount of a composition comprising
(a) reduced coenzyme Q represented by the following formula (1): 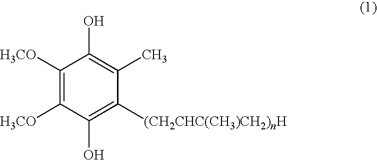
wherein n is an integer of 1 to 12, and
(b) lipoic acid or a derivative thereof,
to the mammal.

15. The method of claim 14, wherein the coenzyme Q is coenzyme Q₁₀.

16. The method of claim 14, wherein the composition further comprises oxidized coenzyme Q represented by the following formula (2): 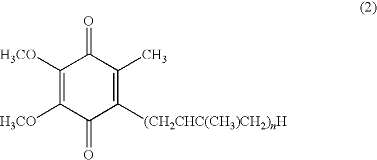
wherein n is an integer of 1 to 12.

17. The method of claim 16, wherein the oxidized coenzyme Q is oxidized coenzyme Q₁₀.

18. The method of claim 14, wherein the lipoic acid is R-lipoic acid.

19. The method of claim 14, wherein the composition further comprises an antioxidant.

20. The method of claim 19, wherein the antioxidant is at least one selected from the group consisting of vitamin C, vitamin C derivatives, probucol, lycopene, vitamin A, vitamin A derivatives, carotenoids, vitamin B, vitamin B derivatives, flavonoids, polyphenols, glutathione, selenium, sodium thiosulfate, vitamin E, vitamin E derivatives, pycnogenol, flavangenol, pyrroloquinoline quinone, pyrroloquinoline quinone derivatives, superoxide dismutase (SOD), glutathione peroxidase, glutathione-S-transferase, glutathione reductase, catalase and ascorbate peroxidase.

21. The method of claim 14, wherein the subject mammal is human.

22. The method of claim 14, wherein the subject mammal is suffering from physical fatigue due to exercise, physical fatigue during or after sickness, or fatigue due to aging.
